(19) United States
(12) Patent Application Publication
(10) Pub. No.: US 2024/0110146 A1
(43) Pub. Date: Apr. 4, 2024

(54) Cell Peeling Device and Cell Peeling Method

(71) Applicant: Hitachi, Ltd., Tokyo (JP)

(72) Inventors: Kota SASAKI, Tokyo (JP); Yuusuke KOURAI, Tokyo (JP); Mutsumi SUZUKI, Tokyo (JP)

(21) Appl. No.: 18/370,981

(22) Filed: Sep. 21, 2023

(30) Foreign Application Priority Data
Oct. 4, 2022 (JP) 2022-160603

(51) Int. Cl. C12M 1/42; C12M 1/34; C12N 13/00

(52) U.S. Cl. CPC C12M 35/04; C12M 41/36; C12N 13/00; C12N 2527/00

(57) ABSTRACT

A cell peeling device includes a vibrator that emits an ultrasonic beam onto a cell aggregate across a container wall, a movement mechanism that moves the vibrator to shift a position thereof relative to a culture container, and a control unit that controls beam power output of the ultrasonic beam and the position of the vibrator, in which the control unit carries out mode switching of switching the beam power output of the ultrasonic beam between a first mode in which the cell aggregate is cut and a second mode in which a peeling portion of the cell aggregate, the peeling portion being formed in the first mode, is peeled from the container wall, and, in the first mode, makes a beam size of the beam power output smaller than a beam size in the second mode while makes an intensity of the beam power output larger than an intensity in the second mode.

### BACKGROUND OF THE INVENTION

### 1. Field of the Invention

The present invention relates to a cell peeling device and a cell peeling method that cut and peel a specific cell aggregate (which will hereinafter be simply referred to as a “cell aggregate” in some cases), such as a cell sheet and an organoid, from a culture surface in a culture container, using ultrasonic waves.

### 2. Description of the Related Art

In the field of medical treatment involving regenerative medicine applications, treatment using cultured cells has been put to practical use. Cells are multiplied in a specific cell culture container (which will hereinafter be simply referred to as “culture container” in some cases), such as a cell culture dish, a cell culture plate, a multi-well plate, a microtiter plate, a flask, a multi-cell culture flask, and a multi-layer cell culture container. Cells in its cultivation process adhere to the bottom of a culture container and grow. The grown cells form an aggregate, such as a cell sheet, which is used for medical treatments and the like.

For example, a technique described in WO 2019/044804 A is known as a method of peeling cells adhering to a culture container, from the container.

WO 2019/044804 A describes a technique of peeling cells by mechanical vibrations created by a piezoelectric element stuck to a cell culture container. According to this technique, mechanical vibrations are applied uniformly to the cultured cells. As a result, the entire cell sheet can be peeled from the culture container in a contactless manner.

### SUMMARY OF THE INVENTION

However, a cell aggregate has its cells adhering to a culture container unevenly because the cells multiply unevenly. Because of this, the technique described in WO 2019/044804 A takes much time to peel the entire cell sheet evenly and is therefore inefficient, which is a problem. Besides, a cell sheet shape that can be peeled off needs to meet restrictive conditions imposed by the shape of the container and a resonance point of mechanical vibrations and therefore forming a cell sheet of any given shape is impossible, which is another problem.

The present invention has been conceived in view of the above problems, and it is therefore an object of the invention to provide a cell peeling device and a cell peeling method that can peel a cell aggregate from a culture container into any given shape in a contactless manner.

In order to solve the above problems, a cell peeling device according to the present invention is provided as a cell peeling device that peels a cell aggregate from a container wall of a culture container, the cell peeling device including: a vibrator that emits an ultrasonic beam onto the cell aggregate across the container wall; a movement mechanism that moves the vibrator to shift a position thereof relative to the culture container; and a control unit that controls beam power output of the ultrasonic beam and the position of the vibrator, in which the control unit carries out mode switching of switching the beam power output of the ultrasonic beam between a first mode in which the cell aggregate is cut and a second mode in which a peeling portion of the cell aggregate, the peeling portion being formed in the first mode, is peeled from the container wall, and, in the first mode, makes a beam size of the beam power output smaller than a beam size of the beam power output in the second mode while makes an intensity of the beam power output larger than an intensity of the beam power output in the second mode.

The present invention provides a cell peeling device and a cell peeling method that can peel a cell aggregate from a culture container into any given shape in a contactless manner.

### BRIEF DESCRIPTION OF THE DRAWINGS

FIG. 1 is a schematic configuration diagram of a cell peeling device according to a first embodiment;

FIG. 2 is a schematic diagram for explaining a first mode and a second mode;

FIGS. 3A and 3B are schematic diagrams for explaining an example of a travel path of an ultrasonic beam;

FIG. 4 is an explanatory diagram of an example of an electric signal;

FIG. 5 is a conceptual diagram of a database according to the first embodiment;

FIG. 6 is a flowchart for explaining the details of a cell peeling method according to the first embodiment;

FIG. 7 is a flowchart for explaining the details of a cell peeling method according to a second embodiment;

FIG. 8 depicts a case where an ultrasonic probe has one piezoelectric element;

FIG. 9 depicts a case where the ultrasonic probe has an acoustic lens; and

FIG. 10 depicts a case where the ultrasonic probe has a plurality of piezoelectric elements.

### DESCRIPTION OF THE PREFERRED EMBODIMENTS

Embodiments of the present invention will hereinafter be described with reference to the drawings. In an embodiment of the present invention, a cell aggregate in a state of adhesion to the inner wall surface of a culture container, the inner wall surface being a culture surface, is peeled off. A cell peeling device according to this embodiment can be used as a part of a cell culture apparatus. A cell peeling method according to this embodiment, for example, is described as a method of forming a culture medium for cells in a generally used cell culture dish to culture adherent cells. A range of application of the present invention is, however, not limited to this method. In addition, this embodiment applies to various adherent cells that can be cultured, and is not limited in application to specific cells. In this embodiment, as a culture medium used to culture cells, both a serum-free culture medium and a culture medium with a serum added thereto can be adopted. The culture container is not limited to a single-layer culture container or double-layer culture container.

To describe this embodiment, a system used in the embodiment is described first. In a state of single-layer culture in which cells immersed in the culture medium in the culture container adhere to its bottom, the system emits an ultrasonic beam from the outside of the bottom of the culture container to let the beam run upward through the culture container. This embodiment, however, is not limited to this system, and may apply also to a system that emits ultrasonic waves from a side face or top face of the culture container to let the ultrasonic waves run through the culture medium.

According to the cell peeling device and cell peeling method of this embodiment, an ultrasonic probe (vibrator) having a piezoelectric element is brought into contact with (stuck to) the outer surface of the culture container, e.g., the outside of its bottom, and an ultrasonic beam whose beam power output is arbitrarily controlled is emitted onto a cell aggregate in its state of adhesion to a culture surface of the culture container. At this time, the ultrasonic probe and the outer surface of the culture container may be in contact with each other via an acoustic coupler for efficient transmission of ultrasonic waves or a matching layer formed of water, silicone rubber, fluororubber, or the like.

According to the cell peeling device and cell peeling method of this embodiment, emitting an ultrasonic beam with a given or greater intensity from the ultrasonic probe applies an acoustic radiation pressure to the cell aggregate. Meanwhile, on a path of the ultrasonic beam, acoustic streaming may be created by the emitted ultrasonic beam. The acoustic radiation pressure and/or the acoustic streaming exerts an upward force on the cell aggregate. It has been experimentally confirmed that emitting an ultrasonic beam with an intensity exceeding a specific threshold (peeling threshold) onto the cell aggregate peels the cell aggregate from the container wall of the culture container. It has also been experimentally confirmed that emitting an ultrasonic beam with an intensity exceeding a threshold larger than the above threshold (cutting threshold) onto the cell aggregate cuts the intercellular junction of the cell aggregate.

In this embodiment, the arbitrarily controlled beam power output of the ultrasonic beam include electronically controlled factors, such as the size (beam diameter) of the ultrasonic beam at a boundary surface between the cells and the culture container, the intensity of the ultrasonic beam, and the transmission time (a duty ratio, repetition intervals, etc.) of the ultrasonic beam. The ultrasonic beam refers to a band-shaped or rod-shaped region formed by ultrasonic waves transmitted from the piezoelectric element.

The intensity of the beam power output of the ultrasonic beam can be controlled, for example, by the following methods. The intensity of a voltage applied to the piezoelectric element of the ultrasonic probe is changed. The wavenumber of a voltage waveform applied (the number of sine waves) is changed. In a case where the ultrasonic probe has a plurality of piezoelectric elements, all or some of piezoelectric elements to which a voltage is applied are selected.

The transmission time of the ultrasonic beam can be controlled, for example, by the following method. The wavenumber or application time of a voltage applied at a specific frequency to the piezoelectric element of the ultrasonic probe is changed. The above specific frequency is determined in accordance with the dimensions and materials of the piezoelectric element, and is set arbitrarily, depending on the piezoelectric element.

The size of the beam power output of the ultrasonic beam can be controlled, for example, by the following methods. In a case where the cell peeling device includes a plurality of ultrasonic probes having piezoelectric elements different in area from each other, the ultrasonic probe is replaced with a more proper ultrasonic probe. In the ultrasonic probe having a plurality of piezoelectric elements, the number of piezoelectric elements to which a voltage is applied is changed. A voltage applied to each piezoelectric element of the ultrasonic probe is delayed by a given delay time to change the focal position of an ultrasonic beam in a transmission axis direction. The frequency of an applied voltage is changed. The distance from an adhesion surface where the cells adhere to the culture container to the ultrasonic probe may be changed.

Control methods of controlling the intensity of the ultrasonic beam, the transmission time, and the beam size have been described above. These control methods are not limited to those described above.

According to this embodiment, based on the shape and coordinate data of the cell aggregate, the beam intensity of the beam power output of the ultrasonic beam is adjusted to a beam intensity exceeding a specific threshold (cutting threshold), and the ultrasonic beam is emitted locally from the outside of the culture container onto the cell aggregate across the container wall to cut the intercellular junction of the cell aggregate.

For example, the ultrasonic probe is moved through a track along the edge of the cell aggregate and is caused to emit an ultrasonic beam onto the cell aggregate. By this process, the cell aggregate can be cut into any given shape. In addition, an ultrasonic beam with a beam intensity equivalent to a peeling threshold lower than the cutting threshold and with a beam size larger than the beam size for cutting is emitted locally. This peels the cell aggregate from the container wall efficiently while keeping the intercellular junction of the cell aggregate intact.

Thus, according to this embodiment, the cell aggregate can be peeled from the culture container into any given shape in a contactless manner. In addition, the cell culture apparatus including the cell peeling device according to this embodiment is able to automatize a cell peeling process with higher throughput.

In this embodiment, for example, the following configuration is disclosed.

A cell peeling device according to this embodiment emits ultrasonic waves onto cells being cultured in a state of adhesion to a culture container to peel off the cells. The cell peeling device includes an ultrasonic probe, a movement mechanism, a voltage applying unit, a control unit, an input unit, a cell state detection unit, a database, and a display unit.

The ultrasonic probe has a piezoelectric element that generates ultrasonic waves by an applied voltage to form an ultrasonic beam. The movement mechanism moves the ultrasonic probe to shift the position of the ultrasonic probe relative to the culture container. The voltage applying unit, which makes up an oscillator, is connected to the piezoelectric element and applies a voltage to the piezoelectric element.

The input unit inputs the shape of an intended area to be cut out from the cell aggregate. The cell state detection unit recognizes the shape, coordinates, and the state of adhesion of the cell aggregate in the culture container. The database holds information on the coordinate data on the cell aggregate, the state of adhesion of the cell aggregate, etc., recognized by the cell state detection unit. The database further holds emission conditions under which an ultrasonic beam is emitted in a first mode or a second mode according to the type of the ultrasonic probe used, the type of the cultured cell, and the number of culture days. The display unit displays at least one of the following things: an actual image obtained by the cell state detection unit, the shape of an area of the cell aggregate, the shape being inputted by the input unit, the mode of an ultrasonic beam being emitted, and an emission position.

The control unit controls the voltage applying unit and the movement mechanism, using information obtained from the input unit, the cell state detection unit, and the database. The control unit carries out mode switching of switching the beam power output of the ultrasonic beam between the first mode for cutting the cell aggregate and the second mode for peeling off the cell aggregate.

The control unit includes a planning unit, a beam condition determining unit, a probe track calculation unit, and a beam controller. The planning unit refers to an emission condition for the ultrasonic beam that is stored in the database, refers to the shape and the state of adhesion of the cell aggregate that are recognized by the cell state detection unit, plans coordinates at which the cell aggregate is cut in the first mode and coordinates at which the cell aggregate is peeled in the second mode, based on the shape inputted by the input unit, and determines an emission condition for emitting the ultrasonic beam in each mode. The beam condition determining unit determines an applied voltage and its waveform, based on the emission condition for the ultrasonic beam determined by the planning unit. The probe track calculation unit calculates a track the probe travels, based on the coordinates planned by the planning unit. The beam controller controls a voltage waveform applied by the voltage applying unit, based on the applied voltage and its waveform that are determined by the beam condition determining unit.

The cell peeling device according to this embodiment may include a plurality of ultrasonic probes that are controlled independently of each other.

### First Embodiment

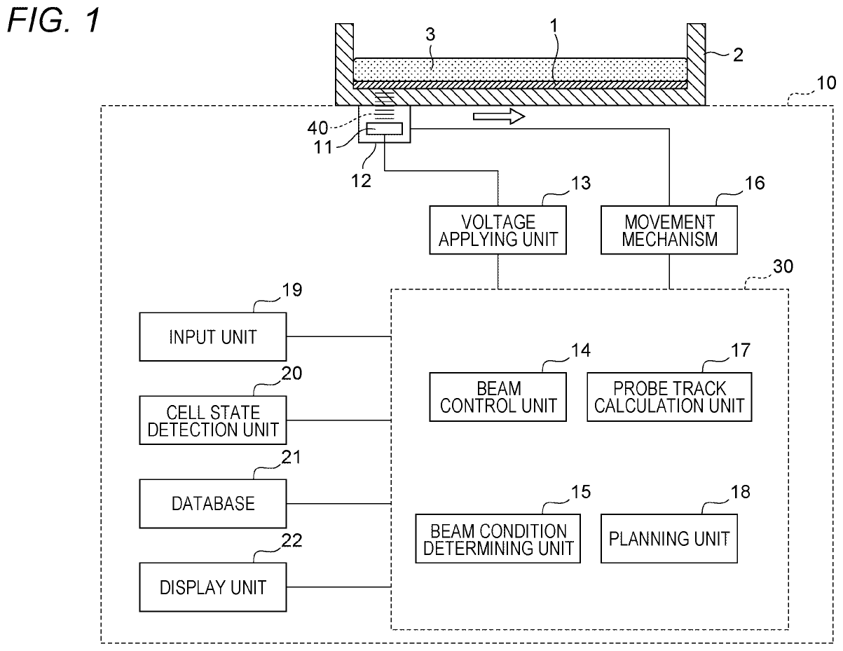

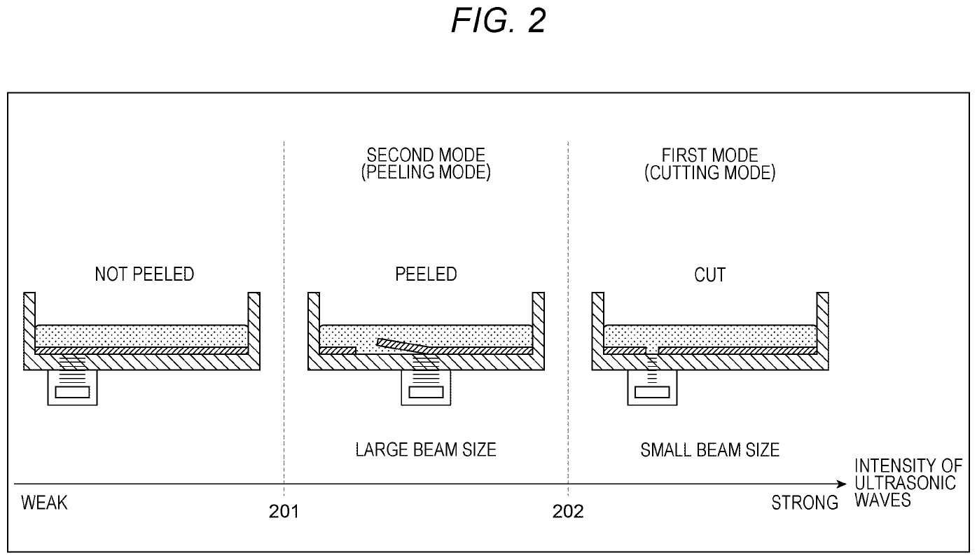

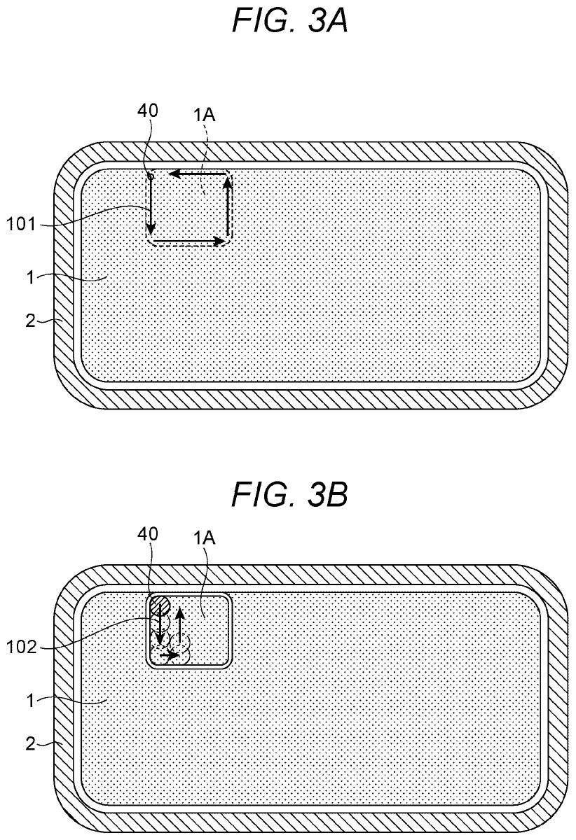

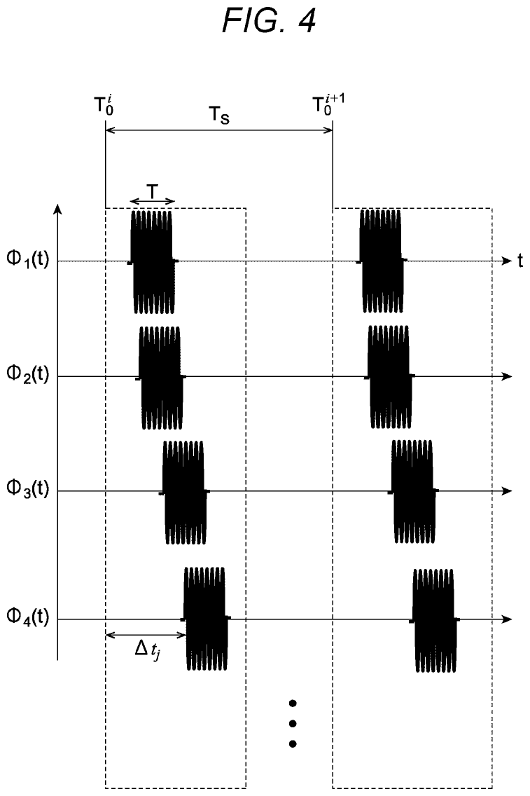

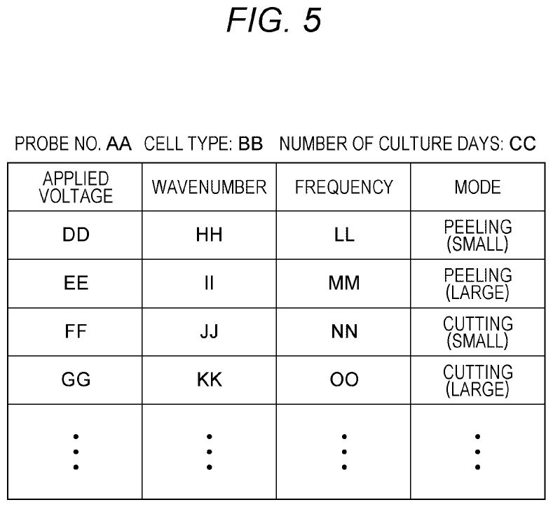

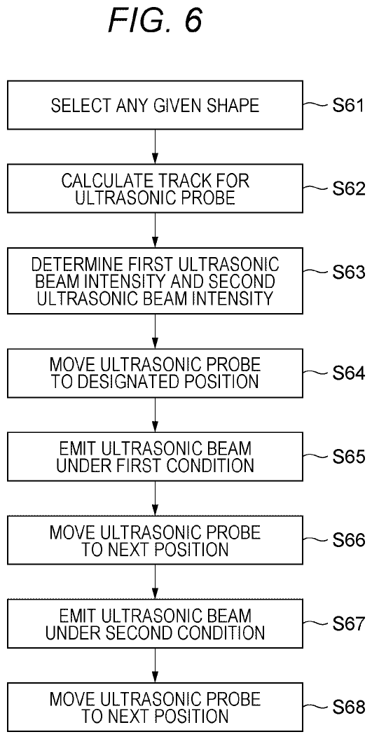

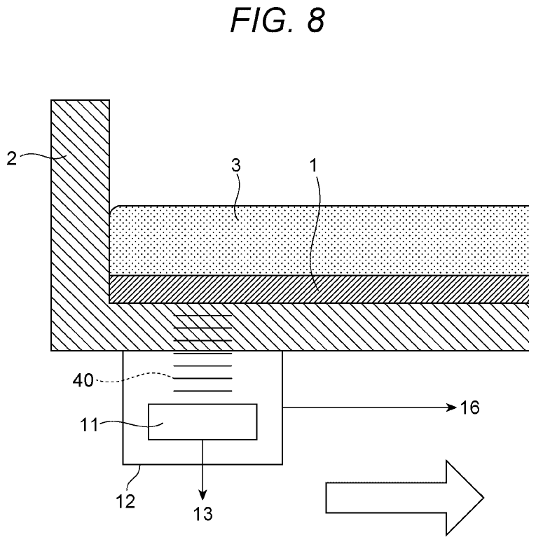

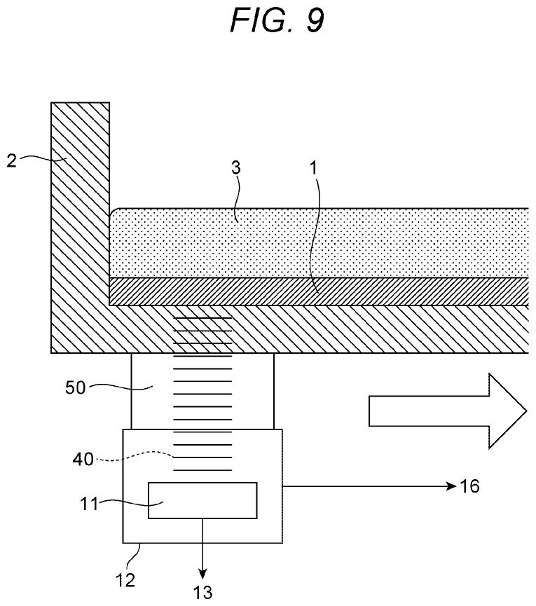

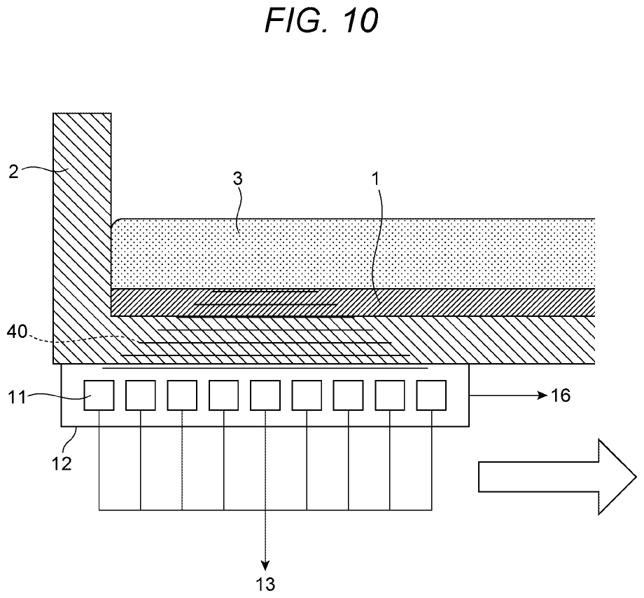

A cell peeling device 10 and a cell peeling method according to a first embodiment will be described with reference to FIGS. 1 to 5. FIG. 1 is a schematic configuration diagram of the cell peeling device 10 according to the first embodiment. FIG. 2 is a schematic diagram showing a relationship between the first and second modes and ultrasonic wave intensity. FIG. 3A is a schematic diagram for explaining an example of a travel path of an ultrasonic beam 40 in a case where the cell aggregate is cut in the first mode. FIG. 3B is a schematic diagram for explaining an example of a travel path of the ultrasonic beam 40 in a case where the cell aggregate is cut in the second mode. FIG. 4 is an explanatory diagram of an example of an electric signal. FIG. 5 is a conceptual diagram of an example of emission conditions for the ultrasonic beam, the emission conditions being stored in the database. FIG. 6 is a flowchart for explaining the details of the cell peeling method according to the first embodiment. FIGS. 8 to 10 are diagrams for explaining configuration examples of an ultrasonic probe 12. FIG. 8 depicts a case where the ultrasonic probe has one piezoelectric element, FIG. 9 depicts a case where the ultrasonic probe has an acoustic lens 50; and FIG. 10 depicts a case where the ultrasonic probe has a plurality of piezoelectric elements.

The cell peeling device 10 is configured to emit the ultrasonic beam 40 onto a cell aggregate 1 immersed in a culture solution 3, such as a culture medium, inside a culture container 2, thereby peeling the cell aggregate 1 from the culture container 2. The cell aggregate 1, which is in the form of, for example, a cell sheet, an organoid, etc., adheres to the inner surface of the bottom of the culture container 2.

The cell peeling device 10 includes the ultrasonic probe 12, a voltage applying unit 13, a movement mechanism 16, an input unit 19, a cell state detection unit 20, a database 21, a display unit 22, and a control unit 30.

The ultrasonic probe 12 is disposed outside the culture container 2, and has one or a plurality of piezoelectric elements 11 that generate an ultrasonic beam (see FIGS. 8 to 10). The ultrasonic probe 12 is brought into contact with a bottom outer surface 2b of the culture container 2, and emits the ultrasonic beam 40 onto the cell aggregate 1 in the culture container 2 across the container wall. In one example, the ultrasonic probe 12 has a plurality of piezoelectric elements 11 of a plate shape. The ultrasonic probe 12 has a ground electrode on the upper surface side of the piezoelectric element 11, that is, on the side of piezoelectric element 11 that is closer to the culture container 2, and has a protective film bonded to the upper surface of the ground electrode. In the ultrasonic probe 12, the protective film is in contact with the outer surface of the culture container 2. The protective film is a thin film made of metal or resin.

The ultrasonic probe 12 has also a signal electrode that applies a voltage to the piezoelectric element 11. The piezoelectric element of a circular shape, elliptical shape, rectangular shape, or the like may be used. The shape of the piezoelectric element, however, is not limited to these shapes. A connection between the signal electrode and a signal line is covered with resin and is fixed.

The piezoelectric element 11 is made of, for example, a ceramic material like lead zirconate titanate and lead titanate, a material like zinc oxide and lithium niobate, or a composite material. The ground electrode and the signal electrode are made of, for example, gold, silver, copper, platinum, titanium, aluminum, and the like.

The ultrasonic probe 12 has a beam varying mechanism (not illustrated) that focuses an ultrasonic beam, the beam varying mechanism being disposed between the ultrasonic probe 12 and the culture container 2. The beam varying mechanism is composed of, for example, one or more acoustic lenses 50 (see FIG. 9). The acoustic lens 50, which is controlled by the control unit 30, changes the focal position of the ultrasonic beam in a transmission axis direction. Output conditions for the ultrasonic beam including focusing conditions for focusing ultrasonic waves by the acoustic lens 50 are determined by the control unit 30. The output conditions may be determined in the following manner: a Fresnel lens that changes the focal point of the ultrasonic beam is provided, a voltage with a modulated frequency is applied to the piezoelectric element 11 of the ultrasonic probe 12, and the output conditions for the ultrasonic beam including the Fresnel lens and the modulated frequency are determined, referring to the state of an end of the cell aggregate, the state being obtained by the cell state detection unit 20. The output conditions may be determined without providing the beam varying mechanism in the following manner: as indicated in FIG. 10, for example, in the ultrasonic probe 12 having a plurality of piezoelectric elements 11, an applied voltage to each piezoelectric element 11 is delayed by a given delay time to change the focal position of the ultrasonic beam 40 in the transmission axis direction, and the output conditions for the ultrasonic beam 40 are determined accordingly.

The voltage applying unit 13 is configured to work as an oscillator that based on a control instruction from the control unit 30, applies a voltage to the piezoelectric element 11 of the ultrasonic probe 12 to cause the ultrasonic probe 12 to generate an ultrasonic beam with intended beam power output. The voltage applying unit 13 applies a voltage to the piezoelectric element 11, for example, in the form of an electric signal shown in FIG. 4. The horizontal axis represents time t and the vertical axis represents examples of time waveforms Φ of the applied voltage to the piezoelectric element. As shown in FIG. 4, the voltage applying unit 13 applies a voltage to the piezoelectric element 11 at a given time T and at a given cycle Ts.

The movement mechanism 16 is a mechanism that moves the ultrasonic probe 12 relative to the culture container 2. In this embodiment, the movement mechanism 16 moves the ultrasonic probe 12 through a track calculated by the probe track calculation unit 17 along the bottom outer surface 2b of the culture container 2. As the movement mechanism 16, a known mechanism, such as an X-Y axis moving table, can be used.

The input unit 19 inputs, for example, data for cutting out a peeling portion of an intended shape from the cell aggregate 1 being cultured that is displayed on the display unit 22. The input unit 19 includes a keyboard and a mouse, with which an operator or the like makes an input operation, and sends input information, such as the shape of the peeling portion, to the database 21 and the control unit 30.

The cell state detection unit 20 detects the shape and the state of adhesion of the cell aggregate 1. As the cell state detection unit 20, for example, an ordinary cell observation device, such as an optical camera or an optical microscope, can be used. The cell state detection unit 20 measures the shape of the cell aggregate 1, end coordinates of the same, or the like, and stores such measurement data in the database 21. To recognize the shape and the state of adhesion of the cells, image processing on an observed cell image may be used.

The database 21 saves (stores) data on the shape (coordinate data) and the state of adhesion of the cell aggregate 1, the shape and the state of adhesion being recognized by the cell state detection unit 20. In addition, in accordance with the type of the ultrasonic probe 12 used, the type of the cultured cells, the number of culture days, and a state of culturing, the database 21 stores emission conditions for the ultrasonic beam in the first mode and the same in the second mode, that is, ultrasonic wave control data necessary for cutting and peeling of the cell aggregate 1, such as the size, intensity, and transmission time of the ultrasonic beam 40.

For example, as shown in FIG. 5, the database 21 stores information on type AA of the ultrasonic probe 12, type BB of the cultured cell, number of culture days CC, applied voltage DD . . . , wavenumber (the number of sine waves) of the ultrasonic beam HH . . . , frequency LL . . . , and mode (first mode, second mode, beam size in each mode). In the first mode and the second mode, multiple beam sizes, such as a small beam size and a large beam size, can be selected according to an area of the cell aggregate 1 that is cut or peeled.

The display unit 22 has a monitor screen, displaying at least one of the following things: actual image obtained by the cell state detection unit 20, the shape of a peeling portion of the cell aggregate, the shape being input by the input unit 19, the mode of ultrasonic waves being emitted, and an emission position.

The control unit 30 includes a beam controller 14, a beam condition determining unit 15, a probe track calculation unit 17, and a planning unit 18, as internal functions implemented by running software on hardware.

The beam controller 14 and the probe track calculation unit 17 calculate a track, through which the ultrasonic probe 12 is moved to cut the cell aggregate 1 into an intended shape and peel the cut shape, and the power output of the ultrasonic beam 40 at a position on the track, and control the movement mechanism 16 and the ultrasonic probe 12.

The control unit 30 determines output conditions for the ultrasonic beam including focusing conditions for focusing of ultrasonic waves by the acoustic lens, and based on the determined output conditions, calculates the beam power output of the ultrasonic beam and the movement track through which the ultrasonic probe 12 is moved by the movement mechanism 16.

The planning unit 18 refers to emission conditions for the ultrasonic beam 40 by accessing the database 21, refers to the shape of the cell aggregate 1 and the state of adhesion of the cell aggregate 1 adhering to the culture container 2, the shape and adhesion state being recognized by the cell state detection unit 20, and based on the shape of the peeling portion inputted by the input unit 19, plans coordinates at which the cell aggregate 1 is cut in the first mode and coordinates at which the cell aggregate 1 is peeled in the second mode.

The beam condition determining unit 15 determines a voltage and its waveform the voltage applying unit 13 applies to the piezoelectric element 11, based on emission conditions for the ultrasonic beam 40 planned by the planning unit 18. Switching the ultrasonic beam to be emitted between the first mode and the second mode may be carried out by software-based means using an electric signal. The ultrasonic beam to be emitted, in another case, may be switched by using a physical button or the like.

The beam controller 14 controls conditions for a voltage applied by the voltage applying unit 13, based on an applied voltage and its waveform determined by the beam condition determining unit 15. From cell state data stored in the database 21, the beam controller 14 reads, for example, the intended shape of the cell aggregate 1 being cultured and a spatial distribution on an end of the cell aggregate 1, and calculates the power output of the ultrasonic beam 40, that is, a voltage signal applied to the ultrasonic probe 12 and the intensity of the voltage signal.

The probe track calculation unit 17 calculates a track for the ultrasonic probe 12 moved by the movement mechanism 16, based on an emission position planned by the planning unit 18. The track for the ultrasonic probe 12, i.e., a trajectory the ultrasonic beam 40 follows, which is calculated by the probe track calculation unit 17, can be set arbitrarily on the input unit 19. Based on the inputted shape, the probe track calculation unit 17 calculates the coordinates of the ultrasonic probe 12 so that it starts from an any given coordinate point and emits the ultrasonic beam 40 along the edge of the inputted shape. After the cell aggregate peels off at an emission site, the probe track calculation unit 17 calculates an amount of move to the next designated position.

As shown in FIG. 3A, the probe track calculation unit 17 calculates a cutting track 101 for cutting the cell aggregate 1 in such a way as to form a peeling portion 1A sectioned off into a desired shape. The cutting track 101 is set so that cutting starts from any given start point and continues along the outer edge of a given shape to draw a continuous loop track.

Subsequently, as shown in FIG. 3B, the probe track calculation unit 17 calculates a peeling track 102 for peeling the peeling portion 1A sectioned by cutting. In a state where the cut peeling portion 1A is already present, the probe track calculation unit 17 calculates an amount of move to the next designated position so that the ultrasonic beam 40 is sequentially emitted along the boundary between the adhesion portion and the peeling portion to peel off the peeling portion 1A. To peel the peeling portion 1A from its end, the probe track calculation unit 17 causes the ultrasonic beam 40 to be emitted on the boundary between a cutting line along which the cell aggregate 1 is cut and the peeling portion 1A demarcated by the cutting line, and after the end of the peeling portion 1A is peeled, calculates a track 102 along which the ultrasonic beam 40 is emitted on the boundary between the resulting peeled area and the adhesion area.

FIG. 2 shows a relationship between the intensity of ultrasonic waves and the first mode and second mode. When the intensity of the ultrasonic beam 40 is smaller than a threshold 201, peeling of the cell aggregate does not occur. When the intensity of the ultrasonic beam 40 is larger than a threshold 202 (first mode), however, the intercellular junction of the cell aggregate at an emission site is broken. When the intensity of the ultrasonic beam 40 is larger than the threshold 201 but is smaller than the threshold 202 (second mode), the cell aggregate 1 is peeled from a container wall 2a as the intercellular junction of the cell aggregate 1 is kept intact.

FIG. 2 shows also a relationship between the beam size and intensity of the ultrasonic beam 40 and the first mode and second mode. In the first mode, the beam power output of the ultrasonic beam is set to power output equivalent to a beam intensity high enough to cut the cell aggregate 1. A cut part of the cell aggregate 1 cannot be used as the cell aggregate 1 for surgery, treatment, etc., and is therefore left wasted. It is therefore preferable that to keep a greater part of the cell aggregate 1 usable, the beam size be as small as possible in the first mode.

In the second mode, on the other hand, it is necessary to peel off the cell aggregate 1 while maintaining its intercellular junction, in which case fewer times of emission of the ultrasonic beam and a smaller intensity of beam power output lead to less destruction of cells. For this reason, a larger beam size is preferable in this mode. Thus, the beam power output of the ultrasonic beam is set such that the beam size is larger in the second mode than in the first mode and that the intensity of the beam power output is smaller in the second mode than in the first mode.

The cell peeling method according to this embodiment will then be described, using a flowchart of FIG. 6. The cell peeling method according to this embodiment will be described exemplarily as a method according to which the cell aggregate 1 is cut in the first mode to form the peeling portion 1A sectioned off into any given shape and the peeling portion 1A is peeled from the container wall 2a of the culture container 2 in the second mode. The cell peeling method according to this embodiment, however, is not limited to the method to be described.

First, as shown in FIG. 6, peeling conditions for the cell aggregate 1 are inputted to the input unit 19. The peeling conditions inputted include, for example, the shape of the peeling portion 1A, the length of its side, and a coordinate point indicating the position of the peeling portion 1A peeled from cell aggregate 1 (selection step S61).

The probe track calculation unit 17 reads the coordinates of an any given shape inputted at step S61 with respect to the origin of the peeling portion LA (which origin is, for example, the center of the culture container 2), the coordinates being stored in the database 21. Subsequently, the probe track calculation unit 17 calculates a track for the ultrasonic probe 12 that corresponds to the shape selected at the selection step S61. The probe track calculation unit 17 calculates the cutting track 101 and the peeling track 102 at an emission point of the ultrasonic beam 40 (track calculation step S62).

Referring to emission conditions for the ultrasonic beam 40 that are set according to the type of the ultrasonic probe 12, the type of the cells of the cell aggregate 1, the number of days of culturing the cells, and the like, the planning unit 18 determines the intensity of the ultrasonic beam in the first mode and the intensity of the same in the second mode, and based on these intensities, determines the first condition and the second condition for an applied voltage to the piezoelectric element 11 of the ultrasonic probe 12 (condition selection step S63).

Subsequently, the movement mechanism 16 changes the relative position of the ultrasonic probe 12 to the culture container 2. Specifically, the movement mechanism 16 moves the ultrasonic probe 12 to a designated position so that the center of the ultrasonic beam 40 is located at a first ultrasonic wave transmission point (origin) (movement step S64). Subsequently, the voltage applying unit 13 applies a voltage to the piezoelectric element 11 of the ultrasonic probe 12 to cause it to generate an ultrasonic wave, thus letting the ultrasonic probe 12 emit the ultrasonic beam 40 onto the cell aggregate 1 under a first condition, that is, with the intensity of the ultrasonic beam in the first mode (S65).

After an exposure portion of the cell aggregate 1, the exposure portion being exposed to the ultrasonic beam 40, is cut by the ultrasonic beam 40 in the first mode, the movement mechanism 16 moves the ultrasonic probe 12 to the next designated position calculated by the probe track calculation unit 17, that is, the next position along the cutting track 101 (S66). This process including steps S64 to S66 is a cutting step defined in the claims.

After the cell aggregate 1 is cut in the first mode along the edge of the inputted given shape (the edge of the peeling portion 1A), the beam controller 14 adjusts the beam power output of the ultrasonic beam 40 to the beam power output conditioned in the second mode. At this time, the intensity of the beam power output of the ultrasonic beam is set smaller than that of the ultrasonic beam in the first mode. In addition, the beam size of the ultrasonic beam 40 is set larger than the beam size of the ultrasonic beam 40 in the first mode (see FIGS. 2, 3A, and 3B). Subsequently, the voltage applying unit 13 applies a voltage to the piezoelectric element 11 of the ultrasonic probe 12 to cause it to generate an ultrasonic wave, thus letting the ultrasonic probe 12 emit the ultrasonic beam 40 onto the cell aggregate 1 under a second condition, that is, with the intensity of the ultrasonic beam in the second mode (S67).

The ultrasonic probe 12 is moved to the next designated position by the movement mechanism 16 so that the ultrasonic beam 40 is sequentially emitted along the boundary between a portion of cell aggregate 1 that has already been peeled off and a portion of cell aggregate 1 that has not been peeled off yet (S68). This process including steps S67 and S68 is a peeling step defined in the claims. In this embodiment, as shown in FIG. 3B, the ultrasonic probe 12 is moved such that the ultrasonic beam 40 follows the track 102 of a zigzag pattern by starting moving straight from a given end of the peeling portion 1A, moving to an adjacent row upon reaching the outer edge of the peeling portion 1A, and turning back and moving straight again. When the entire peeling portion 1A is exposed to the ultrasonic beam 40 and is thus peeled from the container wall 2a, a cell peeling step on one cell aggregate 1 comes to an end.

Thereafter, when another cell aggregate 1 in a state of adhesion is present in one culture container 2 or another cell aggregate 1 is cultured in another well on a multi-well plate, the movement mechanism 16 moves the ultrasonic probe 12 to the next position, that is, to the position of another cell aggregate 1 in one culture container 2 or to the position of another well, where the above series of steps S61 to S68 are executed, by which the peeling portion 1A of any given shape can be peeled from the cell aggregate 1.

In this manner, according to the cell peeling device 10 and the cell peeling method of this embodiment, by emitting the ultrasonic beam 40 onto the cell aggregate 1 being cultured, the cell aggregate 1 can be peeled efficiently from the culture container 2 into any given shape in a contactless manner. In particular, in the first mode in which the cell aggregate 1 is cut, the beam size is reduced while the intensity of the ultrasonic beam is increased. This makes a portion to cut out as small as possible to allow use of a wider area of the cell aggregate 1. In the second mode in which the peeling portion 1A is peeled from the cell aggregate 1, the beam size is increased while the intensity of the ultrasonic beam is reduced. This results in a fewer number of times of emission of the ultrasonic beam onto the cell aggregate 1, thus allowing a reduction in damage to the peeling portion 1A.

### Second Embodiment

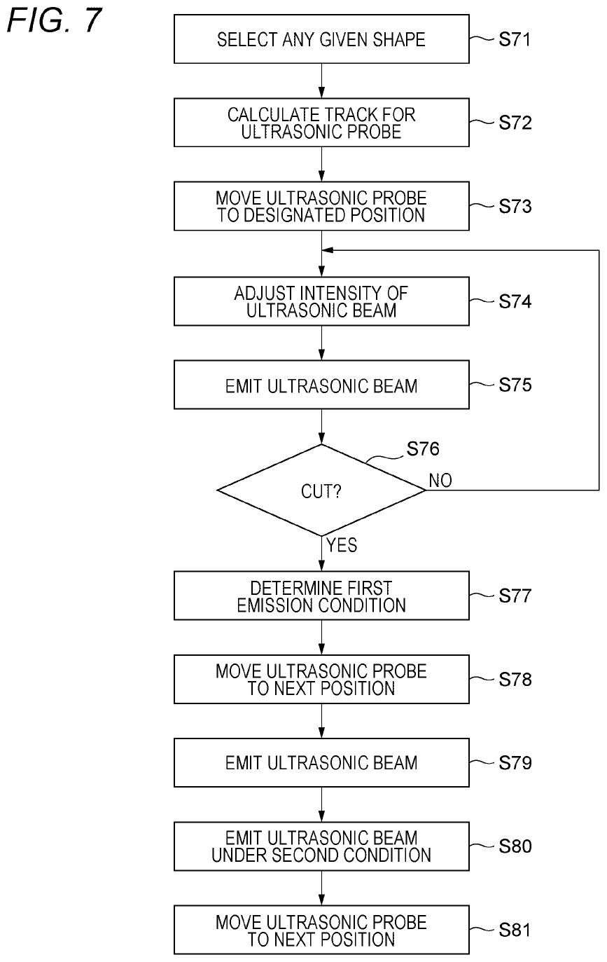

A cell peeling device 10 and a cell peeling method according to a second embodiment will then be described with reference to FIG. 7. FIG. 7 is a flowchart for explaining the details of the cell peeling method according to the second embodiment. The second embodiment will be described, focusing on its differences from the first embodiment.

In this embodiment, the cell aggregate 1 is observed during emission of the ultrasonic beam to evaluate a state of adhesion of the cell aggregate 1 to the container wall 2a, and based on results of the evaluation, thresholds for the intensity of the ultrasonic beam, the thresholds being used in the first and second modes, are determined. According to this embodiment, a cell aggregate whose state of adhesion is uneven because of an on-going culture process can be cut and peeled efficiently with low energy.

As shown in FIG. 7, first, any given shape sectioned off as the peeling portion 1A is selected and is inputted to the input unit 19 (S71). Based on the inputted shape, the probe track calculation unit 17 calculates the cutting track 101 and the peeling track 102 at an emission point of the ultrasonic beam 40 (S72).

Subsequently, the movement mechanism 16 moves the ultrasonic probe 12 to a designated position so that the center of the ultrasonic beam 40 is located at a first ultrasonic wave transmission point (S73). Subsequently, the voltage applying unit 13 applies a voltage to the piezoelectric element 11 of the ultrasonic probe 12 to cause it to generate ultrasonic waves, thus letting the ultrasonic probe 12 emit the ultrasonic beam 40 onto the cell aggregate 1 across the culture container 2 (S75). Following emission of the ultrasonic beam 40, the cell state detection unit 20 detects the cell aggregate 1 being cut or not cut (detection step S76).

When the cell aggregate 1 is cut (YES at S76), the current emission condition is set as a first emission condition (intensity of the ultrasonic beam) in the first mode (S77). The movement mechanism 16 then moves the ultrasonic probe 12 to the next designated position calculated by the probe track calculation unit 17 (S78). Then, the ultrasonic beam is emitted under the first emission condition (S79), which cuts the cell aggregate 1 into any given shape to form the peeling portion 1A.

Subsequently, the ultrasonic beam is emitted onto the peeling portion 1A of the cell aggregate 1 under a second emission condition in the second mode (S80). Under the second emission condition, the intensity of the ultrasonic beam is smaller than that under the first emission condition and is the intensity for peeling the peeling portion 1A, and the beam size is larger than that under the first emission condition. The movement mechanism 16 moves the ultrasonic probe 12 to the next designated position calculated by the probe track calculation unit 17 (S81). Then, emission of the ultrasonic beam 40 and movement of the ultrasonic probe 12 are repeated to peel the peeling portion 1A of the cell aggregate 1 from the container wall 2a.

When an exposure portion of the cell aggregate 1 is not cut by the ultrasonic beam 40 emitted at S75 (NO at S76), the beam controller 14 recalculates and adjusts conditions for the ultrasonic beam 40 to transmit, such as the intensity and the transmission time of the ultrasonic beam 40 (S74). Thereafter, the voltage applying unit 13 applies a voltage for re-emission of the ultrasonic beam 40 under the recalculated conditions (return to S75). In other words, when the cell aggregate 1 being not cut is detected at step S76, the threshold 202 for the first emission condition is specified as the power output of ultrasonic waves is gradually increased until the cell aggregate 1 is cut. The process including steps S74 to S76 corresponds to a specifying step defined in the claims.

In this embodiment, whether the cell aggregate 1 has been cut is checked immediately after emission of the ultrasonic beam 40 onto the cell aggregate 1. When the cell aggregate 1 is not cut, conditions for the ultrasonic beam 40 are immediately recalculated (the intensity is increased) and the ultrasonic beam 40 under the recalculated conditions is emitted on the same part again. The recalculated conditions for the ultrasonic beam 40 include, for example, the longer transmission time of the ultrasonic beam 40 and the higher transmission output of the same.

The method described in this embodiment is the method according to which whether the cell aggregate 1 being cultured has been cut is checked at each transmission point of the ultrasonic beam 40 and the first emission condition is determined on condition that the cell aggregate 1 being cut is confirmed. However, the cell peeling method is not limited to this method.

For example, the beam controller 14 calculates the intensity and transmission time of the ultrasonic beam 40, the intensity and transmission time being read from the database 21, and the coordinates of the ultrasonic beam 40, and creates plan data on ultrasonic wave transmission conditions in advance so that the ultrasonic beam 40 is emitted onto any given emission point. Then, according to the plan data, the voltage applying unit 13 and the movement mechanism 16 cause an intended ultrasonic beam 40 to be emitted on an intended position, and after emission of the ultrasonic beam 40 is executed according to the plan data, the cell state detection unit 20 checks whether the cell aggregate 1 is cut at an exposure portion. Subsequently, plan data for emitting the ultrasonic beam again onto an uncut portion in its state of adhesion is created, and the ultrasonic beam 40 is emitted according to the plan data to cut the cell aggregate 1 (cell sheet). These processes may also be adopted as another method.

Some embodiments of the present invention have been described above. These embodiments have been presented as examples and are not intended to limit the scope of the invention. These novel embodiments may be implemented in various other forms, and various omissions, substitutions, and changes of their constituent elements may be made without departing from the substance of the invention. These embodiments and modifications thereof are included in the scope and substance of the invention and are included also in the claims describing the invention and the equivalent of the claims.

What is claimed is:

1. A cell peeling device that peels a cell aggregate from a container wall of a culture container, the cell peeling device comprising:
a vibrator that emits an ultrasonic beam onto the cell aggregate across the container wall;
a movement mechanism that moves the vibrator to shift a position thereof relative to the culture container; and
a control unit that controls beam power output of the ultrasonic beam and the position of the vibrator, wherein
the control unit carries out mode switching of switching the beam power output of the ultrasonic beam between a first mode in which the cell aggregate is cut and a second mode in which a peeling portion of the cell aggregate, the peeling portion being formed in the first mode, is peeled from the container wall, and, in the first mode, makes a beam size of the beam power output smaller than a beam size of the beam power output in the second mode while makes an intensity of the beam power output larger than an intensity of the beam power output in the second mode.

2. The cell peeling device according to claim 1, comprising a cell state detection unit that detects coordinates of the cell aggregate and a state of adhesion of the cell aggregate to the container wall, wherein
the control unit determines the beam power output with which the ultrasonic beam is emitted from the vibrator and a track through which the vibrator is moved, based on the coordinates of the cell aggregate and on the state of adhesion of the cell aggregate to the container wall.

3. The cell peeling device according to claim 1, comprising a database storing an output condition for the beam power output of the ultrasonic beam in accordance with a type of the cell aggregate and a type of the culture container, wherein
the control unit determines the beam power output with which the ultrasonic beam is emitted from the vibrator and a track through which the vibrator is moved, based on the type of the cell aggregate and the output condition for the beam power output of the ultrasonic beam, the type and the output condition being stored in the database.

4. The cell peeling device according to claim 1, wherein
the vibrator includes a beam varying mechanism that focuses the ultrasonic beam.

5. The cell peeling device according to claim 4, wherein
the beam varying mechanism includes one or more acoustic lenses that focus the ultrasonic beam, and wherein
the control unit determines an output condition for the ultrasonic beam, the output condition including a focusing condition for focusing an ultrasonic wave by the acoustic lens.

6. The cell peeling device according to claim 4, wherein
the beam varying mechanism includes a Fresnel lens that changes a focal point of the ultrasonic beam, and wherein
the control unit determines an output condition for the ultrasonic beam, the output condition including a voltage that is applied to a piezoelectric element of the vibrator, with a frequency of the voltage modulated.

7. The cell peeling device according to claim 1, wherein
the vibrator includes a plurality of piezoelectric elements, and wherein
the control unit delays an applied voltage to each of the plurality of piezoelectric elements of the vibrator by a given delay time to change a focal position of the ultrasonic beam in a transmission axis direction.

8. The cell peeling device according to claim 1, comprising a display unit that displays at least one of an actual image of the cell aggregate, a shape of the cell aggregate, a mode of the ultrasonic beam, a track, and an emission position.

9. The cell peeling device according to claim 2, wherein
an end detected by the cell state detection unit is peeled by emitting the ultrasonic beam onto the end, and then the ultrasonic beam is emitted onto a boundary between a peeled area of the cell aggregate and an adhesion area of the cell aggregate.

10. A cell peeling method of peeling a cell aggregate from a culture container to which the cell aggregate being cultured adheres, the cell peeling method comprising:
a selection step of selecting a shape of a peeling portion to be peeled from the cell aggregate;
a track calculation step of calculating a track for a vibrator, the track corresponding to the shape selected at the selection step;
a condition selection step of determining a first condition and a second condition for a beam intensity of an ultrasonic beam in accordance with the cell aggregate and the culture container;
a movement step of moving the vibrator to a designated position determined at the track calculation step;
a cutting step of applying a voltage to a piezoelectric element included in the vibrator under the first condition to cause the vibrator to generate an ultrasonic wave and emit an ultrasonic beam; and
a peeling step of applying a voltage to a piezoelectric element included in the vibrator under the second condition to cause the vibrator to generate an ultrasonic wave and emit an ultrasonic beam.

11. A cell peeling method of peeling a cell aggregate from a culture container to which the cell aggregate being cultured adheres, the cell peeling method comprising:
a selection step of selecting a shape of a peeling portion to be peeled from the cell aggregate;
a track calculation step of calculating a track for a vibrator, the track corresponding to the shape selected at the selection step;
a movement step of moving the vibrator to a designated position determined at the track calculation step;
an ultrasonic beam emitting step of applying a voltage to a piezoelectric element included in the vibrator to cause the vibrator to generate an ultrasonic wave and emit an ultrasonic beam;
a detection step of detecting the cell aggregate being cut or not cut at a part on which the ultrasonic beam is emitted at the ultrasonic beam emitting step;
a specifying step of specifying a cutting threshold while gradually increasing power output of an ultrasonic wave until the cell aggregate is cut, the specifying step being executed when the cell aggregate being not cut is detected at the detection step;
a cutting step of applying a voltage under a first condition specified at the specifying step to generate an ultrasonic wave and emit the ultrasonic beam; and
a peeling step of applying a voltage to a piezoelectric element included in the vibrator under a second condition to cause the vibrator to generate an ultrasonic wave and emit the ultrasonic beam.
